(19) United States
(12) Patent Application Publication
(10) Pub. No.: US 2025/0204955 A1
(43) Pub. Date: Jun. 26, 2025

(54) THERAPEUTIC DEVICES AND METHODS FOR REDUCING INJURY DURING ARISING FROM LABOR AND DELIVERY

(71) Applicant: Materna Medical, Inc., Mountain View, CA (US)

(72) Inventors: Mark Juravic, San Francisco, CA (US); Michael Stewart, San Jose, CA (US); Lisa Molloy, Mountain View, CA (US); Kelly Ashfield, Mountain View, CA (US); Connor Wison, Mountain View, CA (US); Stephen Kmak, Mountain View, CA (US)

(73) Assignee: Materna Medical, Inc., Mountain View, CA (US)

(21) Appl. No.: 18/394,218

(22) Filed: Dec. 22, 2023

(51) Int. Cl. A61B 17/42; A61B 17/00; A61B 17/02; A61B 90/00; A61B 90/50

(52) U.S. Cl. CPC A61B 17/42; A61B 17/02; A61B 90/50; A61B 2017/00862; A61B 2090/032

(57) ABSTRACT

The systems, devices and methods to reduce trauma and injury to tissue during labor and delivery. In particular, the systems and methods relate to devices that retain dilation support devices, dilation devices, in place in the vaginal canal during the certain stages of labor. These devices retain the dilation device in the vaginal canal at therapeutically effective locations and maintain the dilation device in such positions for periods of time sufficiently long to allow the dilation device to have a therapeutic effect.

### FIELD OF THE INVENTION

The field is labor and delivery and more particularly, improved devices and methods for addressing discomfort and tissue damage during labor and reducing pelvic dysfunction caused by certain aspects of labor and delivery.

### BACKGROUND OF THE INVENTION

Approximately 134 million women give birth worldwide each year, of whom 3.0 million give birth vaginally in the United States.

Birth most commonly happens through the process of labor and delivery. Labor is the physiologic process by which a mother pushes the fetus from the uterus to the outside world. Labor is characterized by regular, painful contractions with change in cervical dilation and effacement. The contractions drive the fetus through the birth canal, which includes the cervix and the vaginal canal. The birth canal provides a passage for the fetus through the pelvic floor of the mother.

The pelvic floor is an important and complex connection of muscles, ligaments and bone structure which act as a support for the pelvic organs such as the bladder, the rectum, intestines and urethra. As such, the passage of the fetus from the uterus to the outside world requires a passage through the dense complex of muscles of the pelvic floor. The passing of the fetus may adversely affect the health of this complex of muscle that is critical to daily activities. Injury to this complex of muscle may lead to pelvic floor disorders which are conditions that, among other things, affect the proper functioning of a woman's pelvic organs.

Although there is a known link between labor and delivery and pelvic floor disorders, the exact physiologic mechanisms that lead to many of these disorders remain under investigation. It is thought that damage to the pelvic floor musculature during childbirth may be one of the most significant causes of pelvic floor disorders. Babies can be relatively big, with large heads and broad shoulders, as compared to the diameter of the birth canal. This, as is generally known, can lead to injury of the mother, and in particular to the pelvic floor of the mother, as the mother pushes the baby through the birth canal so fetal descent takes place.

During vaginal delivery, labor and delivery are clinically divided into three stages. The first stage begins with cervical effacement and is completed with full cervical dilation that will allow the passage of the fetus through the birth canal. The second stage is defined by the passage of the fetus through the birth canal, described as the Cardinal Movements of Labor. During the second stage the fetus descends as it moves and rotates to overcome the resistance presented by the maternal bony pelvis and the soft tissue of the pelvic floor. The second stage ends with the delivery of the fetus. The third stage is comprised of the separation and expulsion of the placenta.

As the fetal head descends into the birth canal during the second stage, it exerts direct pressure on the pelvic floor. The fetus passes through the birth canal by stretching the fibers of the levator ani muscle and connective tissues, drastically distending and often damaging the pelvic floor musculature. Unlike the gradual dilation of the cervix during the first stage of labor, the pelvic floor and vaginal canal must dilate rapidly during the second phase of labor. The introitus, or opening of the vagina, for the average woman, has a baseline resting diameter of roughly 2.6 cm. During the first phase of labor, while the cervix is dilating, the vagina stays at this baseline diameter, but when the baby passes through the cervix the vaginal canal dilates from 2.6 cm up to 10 cm, the average head diameter of a newborn baby. This 3.8× expansion in diameter and approximately 15× expansion in area occurs in a matter of seconds to minutes.

Studies show that 15-35% of labor processes, so in some years more than one third of women in labor, suffer damage to their pelvic floor muscles, where the mother's muscles are torn from the pelvic bone or over-stretched to the point where they become functionally impaired. The tearing of the muscle from the supporting bone, termed an avulsion, is a significant injury and complete avulsions are strongly associated with the development of pelvic organ prolapse later in life. Repairing these injuries is invasive and the repair procedures can also be ineffective. Further, physicians also recognize that the financial costs of treating such injuries to the mother can be substantial.

One system for reducing trauma during vaginal delivery, which can be understood for this discussion as all the soft tissue involved during the second phase of labor and delivery, is disclosed in U.S. Pat. No. 9,492,197 issued to the assignee hereof. Such systems can reduce tissue damage and injury to the patient and offer a useful procedure to offer improved patient recovery and reduce unwanted indications later in life arising from or exacerbated by tissue injury during labor and delivery. Although such systems as disclosed in U.S. Pat. No. 9,492,197 can work well, there remains a need to make use of such systems for facile for patients and clinicians.

### SUMMARY OF THE INVENTION

The systems, devices and methods described herein reduce trauma and injury to tissue during labor and delivery. In particular, the systems and methods described herein relate to devices that retain dilation devices in place in the vaginal canal during the second stage of labor. These devices retain the dilation device in the vaginal canal at therapeutically effective locations and maintain the dilation device in such positions for periods of time sufficiently long to allow the dilation device to have a therapeutic effect. Further, such devices may allow dilation devices to be retained in place in the vaginal canal as the patient moves through the common positions of labor and moves about the patient's bed.

Dilation devices such as those manufactured and sold by the assignee hereof, can therapeutically treat tissue of the vaginal canal during labor, and in particular, before the fetus enters the vaginal canal. The dilation device can, essentially slowly apply an expanding force to the tissue wall of the vaginal canal thereby incrementally and therapeutically loosening the tissue of the vaginal canal and allowing it to expand elastically, reducing tearing of the tissue and separating of the tissue from the pelvis. However, as women's bodies are generally designed to push babies out during childbirth it may be difficult for a dilation device to remain in the vaginal canal during labor, as the biomechanical forces arising during the second stage of labor tend to push objects from the vaginal canal. The systems, devices and methods described herein retain the dilation device within the vaginal canal for a period of time sufficient to achieve a therapeutic effect, such as for example two hours, one hour, fifty minutes, forty-five minutes, thirty minutes, or some other time, greater or less than an hour and determined by the treating clinician as appropriate for the patient under treatment and in labor and delivery.

In one embodiment, the devices include an adapter disposed at the proximal end of the vaginal canal dilation device, sometimes referred to herein as a dilation device or a dilator. The adapter may be shaped to releasably engage with the proximal end of the dilation device and to allow the distal end of the dilation device to extend distally from the adapter. The releasable engagement of the adapter allows the adapter to be connected to and disconnected from the proximal end of the dilation device during the labor process. In one typical embodiment the adapter is sized to fit snugly over at least a portion of the proximal end of the dilation device, to thereby provide a frictional engagement that creates a mechanical connection between the adapter and the proximal end of the dilation device. The frictional engagement is sufficiently strong to maintain the adapter on the proximal end of the dilation device during movement of the patient of the type typically expected during the labor and delivery process. This can include rolling over onto the patient's side, sliding the buttocks of the patient across the surface of the bed and perhaps even leaving the bed and standing upright or squatting down during portions of the second phase of labor. In any case, the frictional engagement is sufficiently strong to allow useful attachment of the adapter to the proximal end of the dilation device and to resist the clinically expected forces such that the adapter does not readily disengage from the dilation device during the labor process.

In alternate embodiments the adapter is integral to the distal end of the dilation device. In such alternate embodiments, the distal end of the dilation device may be formed of a plastic material and it may be shaped to provide the distal end of the dilation device and may have integrated into the shape an adapter suitable for use with the systems and methods described herein.

In certain embodiments, the adapter has mounts for receiving a retention harness. Typically, the mounts are outwardly extending arms that provide a recess for receiving the retention harness thereby allowing the retention harness to mechanically engage with the adapter. The retention harness may be made of fabric or some flexible material and it will have a length that is sufficient to wrap around a thigh of the patient and have a section that traverses the thigh of the patient at a location that is superior to the pubic arch of the patient. Typically, the retention harness is adjustable to accommodate patients of different size and body-type. The retention harness has securing features that are shaped to engage with the attachment features of the adapter. Typically, the retention harness has a fastening mechanism that may be employed to secure the retention harness to the body of the patient. The fastening mechanism is typically releasable and in certain embodiments comprises hook and loop fastener to provide a frangible release mechanism allowing for ready release from the body of the patient.

More particularly, the systems and methods described herein include in one aspect, a device for reducing injury to a patient during a labor and delivery procedure, comprising an adapter shaped to engage with a proximal end of a vaginal canal dilation device and to allow the distal end of the dilation device to extend distally from the adapter and having mounts for receiving a retention harness, and a retention harness made of flexible material and having a length sufficient to wrap around a thigh of the patient and having a section that traverses the thigh of the patient at a location superior the pubic arch of the patient and having securing features that are shaped to engage with the attachment features of the adapter, whereby the retention harness restricts the adapter from moving proximally away from the birth canal of the patient.

Optionally, the retention harness comprises one or more straps of elastic material. Further optionally, the one or more straps of elastic material include a fastener to allowing the one or more straps to form a closed loop around the body of a patient. Further it may be that the one or more straps include a hook and loop fastener for allowing the one or more straps to form a closed loop around one or more thighs of the patient.

In some common embodiments, but not always, the retention harness comprises one strap of flexible material sized to wrap around the waist and around the thighs of the patient to form a harness having two sections of strap positioned between the thighs of the patient and superior to the pubic arch of the patient.

In some common embodiments, but not always, the retention harness has a release that disconnects the harness from the patient or the adapter upon application of a proximally directed force that exceeds an expected force of a baby traveling proximally through the birth canal during delivery.

Optionally, the release comprises hook and loop fastener arranged on the strap to provide a frangible release for disconnecting the harness from the patient or the adapter.

In some common embodiments, the mounts of the adapter comprise arms extending radially and proximally from the adapter to form a bend that is sized to engage with the retention harness. Optionally, the arms are formed as rails that attach to the sides of the adapter and provide an interior slot for receiving the retention harness. Often, but not always, the adapter comprises a plastic body shaped to fit around a handle of the dilation device. But optionally, the adapter may be an integral part of the vaginal canal dilation device, so it is built into the dilation device and part of that device. Optionally, the arms snap into the dilation device, and in that way are integral with the dilation device. In other embodiments, the arms may be molded as part of the proximal end of the dilation device.

In another aspect, there are provided methods for reducing tissue injury during a labor and delivery procedure, comprising providing a vaginal canal dilation device of the type used to provide a therapeutic expansive force during at least a portion of the labor procedure, providing a wearable retention harness having a length sufficient to wrap about a waist and at least one thigh of the patient, securing the retention harness to the patient by wrapping the harness around the waist and at least one thigh of the patient so that the harness secures to the patient at a first location proximate the pubic arch and at a second location superior the pubic arch, and securing the dilation device into the retention harness at the second location and disposing at least a portion of the dilation device within the vaginal canal and securing a different portion of the dilation device to the retention harness such that the retention harness resists unwanted exit of the dilation device from the vaginal canal.

Optionally, the methods may include providing an adapter coupled to a proximal end of a vaginal canal dilation device to allow the distal end of the dilation device to extend distally from the adapter and having mounts for slidably receiving the retention harness. This may include providing an adapter includes providing a removable adapter capable of releasably coupling to the proximal end of a vaginal canal dilation device and optionally may include providing a vaginal canal dilation device having a proximal end with the mounts integrated therein.

In some practices, securing the retention harness to the patient includes employing a fastener to fasten a first section of the retention harness to a second section of the retention harness. Employing a fastener may include employing at least one of a hook and loop fastener, a button, a zipper, a clip and a buckle.

Typically, the method secures the vaginal canal dilation device into the retention harness at the second location and disposes at least a portion of the dilation device within the vaginal canal to secure the retention harness and to secure the dilation device at a location within the vaginal canal of the patient so that an expansion force may be applied to the levator ani muscles of the patient during the labor process.

The method may include applying force at a location and for a time, either a continuous time or incremental time, that has a therapeutic effect and may include maintaining the vaginal canal dilation device within the vaginal canal of the patient and applying an expansion force to a tissue wall of the vaginal canal for a period of time between 30 minutes and 120 minutes. Optionally, the method may include temporarily removing the vaginal canal dilation device from the vaginal canal during the period of time between 30 minutes and 120 minutes.

### BRIEF DESCRIPTION OF THE DRAWINGS

FIG. 1 illustrates one embodiment of a retaining device for securing in place a vaginal dilation device during a labor and delivery process;

FIGS. 2a-2f depict in more detail the structure of two example dilation devices for therapeutically expanding the vaginal canal prior to passing the head of the fetus;

FIGS. 3a-3e depict embodiments of an adapter for securing to the proximal end of a dilation device, such as the example dilation device depicted in FIG. 1;

FIG. 4 depicts on embodiment of a retention harness;

FIGS. 5a-5f depict one example of a retention harness being secured to a model patient;

FIGS. 6a and 6b illustrate the retention device in place around the habitus of a patient and retaining a dilation device in place for therapeutic treatment;

FIGS. 7a-7d depicts example labor positions;

FIGS. 8a and 8b illustrate an uninjured and injured pelvic floor and the type of injury that the devices described herein reduce;

FIGS. 9a and 9b depict the dilation device at the beginning of a treatment and toward the end of a treatment;

FIG. 10 depicts one example treatment process;

FIGS. 11a and 11b depict an alternative embodiment of an adapter;

FIG. 12 depicts an alternative embodiment of an adapter;

FIGS. 13a and 13b depict an alternative embodiment of an adapter;

FIG. 14 depicts an alternative embodiment of a retention device of the type described herein;

FIG. 15 depicts a further alternative embodiment of a retention device of the type described herein;

FIG. 16 depicts the retention device of FIG. 15 partially positioned about the body of a model patient; and

FIG. 17 depicts the retention device of FIG. 15 secured to a dilation device and positioned about the body of a model patient.

### DETAILED DESCRIPTION

The devices and methods described herein are intended to prepare and dilate vaginal tissue and perineal tissue during labor to reduce or prevent, among other things, pelvic floor damage and later development of pelvic floor disorder. In particular, the devices and methods described herein provide for therapeutically treating a mother during early stages of labor in preparation for the delivery phase of labor. To this end, in some embodiments, the devices include retention devices that can retain a dilation device within the vaginal canal of the mother during the early stages of labor, preferably doing so in a manner that allows for the mother to move through some of the typical labor positions employed during the labor and delivery process. The retention devices described herein may by removable to allow a clinician to casily remove the retention device from the patient and optionally remove the dilation device as well, as the clinician needs or prefers during treatment. Additionally, the retention devices are designed to be facilely reapplied to the patient to allow the patient to continue the dilation treatment process during labor. Thus, as described herein the retention devices allow for maintaining a dilation device at a proper location and for a time period, whether a continuous time period or a time period arising from intermittent treatment periods, sufficient to achieve a therapeutic effect, while also allowing for the ready and facile removal of the retention device and the dilation device from the patient to allow the clinician to attend to different aspects of the labor and delivery process.

In certain embodiments, the devices comprise retention devices that include an adapter that couples to the distal end of a dilation device and a retention harness that couples to the adapter and retains the adapter and therefore the coupled dilation device in place during the labor process. Typically, the retention device retains the dilation device in the patient's vaginal canal at a location and for a time period therapeutically sufficient to prepare for passage of the baby during the birthing process. For example, the retention devices described herein may retain the dilation device at a location and for a time period that is sufficient to stretch the patient's vaginal canal tissue until, for example, the diameter of the vaginal introitus of the patient has reached a diameter of approximately 10 cm, roughly the size of the fetal head. In some embodiments, the retention device retains the dilation device such that the distal end of the dilation device enters the vaginal introitus and extends into the vaginal canal for the first 3-4 cm of the vagina, and maintains the dilation device at this location to gradually expand the vagina from a resting diameter of 2-3 cm to a fully expanded diameter equal to the size of the delivering fetus, approximately 10 cm. The retention devices described herein may work with dilation devices that expand either manually or via an automatic actuation system, and the dilation device can be quickly collapsed and removed if needed. Typically, the retention device retains the dilation device at the proper anatomical location for a duration on the order of one to three hours, although the time of treatment may vary and be longer or shorter and may be set by the treating clinician. As during this time, clinicians may need to inspect the progress of labor and the mother may need to move about the delivery room, the retention device may be removable for periodic separation of the dilation device from the mother.

The retention devices described herein can be attached to the patient during some part, or all of, the first phase of labor and removed prior to delivery during the second phase of labor, allowing the second phase to proceed. The retention devices may be used prior to the second phase of labor, so prior to the baby passing through the birth canal and prior the baby contacting the dilation device. Depending on how long it takes to achieve sufficient therapeutic vaginal dilation, such as for example approximately 10 cm, the dilation device can be inserted at some point during the first phase of labor. The dilation diameter target of 8 cm means that the device should be used in a hospital under the supervision of trained obstetricians and nurses. The dilation device and the retention device can be used without any anesthesia, or under local anesthetic. The tissue can also be prepared after administering an epidural, or other analgesic, which would eliminate any pain or discomfort the device may cause. Local anesthesia can also be placed on the tissue contacting surfaces of the device to minimize pain.

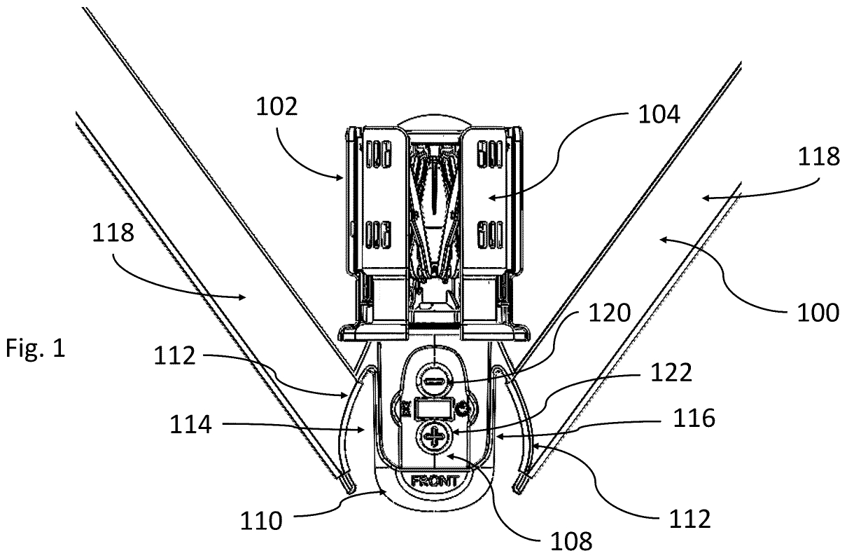

FIG. 1 depicts one embodiment of a retention device 100 of the type described herein. As shown in FIG. 1, the retention device 100 includes an adapter 110 and a retention harness 118. The adapter 110 engages with and attaches to a proximal end 108 of a dilation device 102 allowing the distal end 104 of the dilation device 102 to extend outwardly and distally from the adapter 110, along the longitudinal axis (not marked) of the dilation device 102. One example of a dilation device that may be used with the retention system and methods described herein is disclosed in U.S. Pat. No. 9,492,197 issued to the assignee hereof.

The adapter 110 includes a main body 116 and two arms 112. In the depicted embodiment the main body of the adapter is generally conically shaped, with one side to the conical shape being open to allow access to the front face of the dilation device. The two arms 112 attach at a distal point of the main body 116 and extend radially outward from the main body 116 and generally along the longitudinal axis of the main body 116 toward the proximal end of the main body 116. The arms 112 create two recesses 114 between the two arms 112 and the main body 116. A retention harness of flexible material 118 fits into the recesses 114. Although not shown, the retention harness 118 depicted in FIG. 1 has a length sufficient to wrap around the thigh of a patient at a location that is superior to the pubic arch of the patient. This provides a point of mechanical connection that is, at least at one point of contact, superior to the proximal end of the vaginal canal. As contractions may push the dilation device proximally and out of the vaginal canal, the attachment superior to the pubic arch provides a connection for the retention harness that secures the harness superior to the pubic arch. This connection allows the retention harness to apply a force to the adapter that opposes a force tending to push the dilation device from the vaginal canal and tends to retain the dilation device in the vaginal canal.

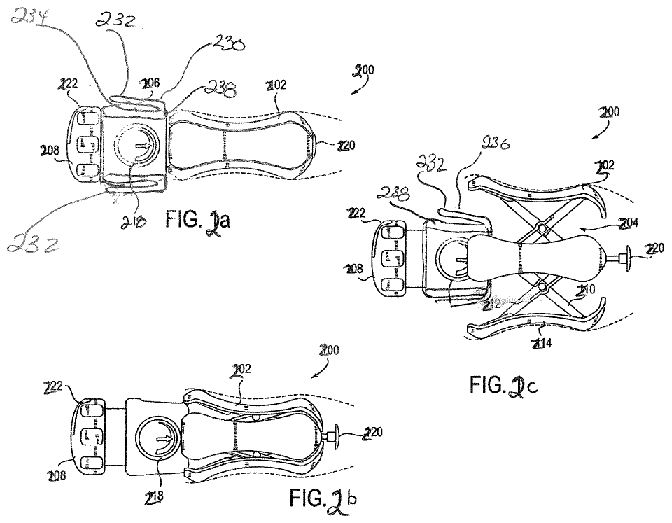

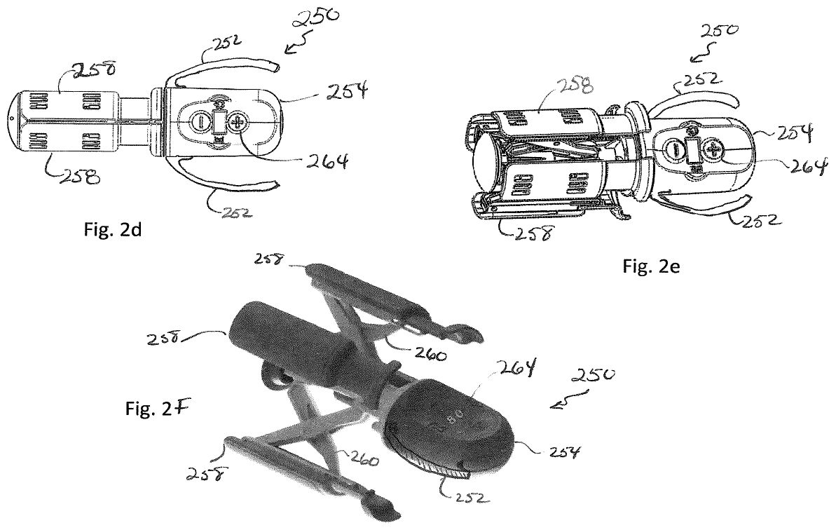

The depicted retention harness 118 is sized and shaped to secure to the body of the patient and hold the adapter 110, with the engaged dilation device 102, in place during labor, or at least for a portion of the labor process. The dilation device 102 therapeutically treats the tissue of the vaginal canal prior to travel of the fetus through the vaginal canal and toward the introitus. In the embodiment depicted in FIG. 1, the dilation device 102 is an electric motor operated dilation device that includes an electric motor for expanding the dilation device 102 from a contracted configuration (shown in FIG. 1) to an expanded configuration. Such motorized dilation devices 102 can expand based on a clinician accessing a user interface control, such as controls 120 and 122. Such controls can cause the dilation device to activate the electric motor and cause the dilation device to expand or contract. An alternate dilation device that is manually operated and includes a rotatable knob to cause the dilation device to expand or contract is depicted in FIGS. 2a-2c. One example of a dilation device on the type depicted in FIGS. 2a-2c is disclosed in U.S. Pat. No. 9,492,197 assigned to the assignee hereof.

In the example of FIGS. 2a-2c, the dilation device 200 is a vaginal dilation device that includes rigid or semi-rigid pads 202, arms 204, handle 206, and expansion mechanism 208. In the depicted example, the dilation device 200 includes four sets of pads 202 and arms 204. In other embodiments, a different number of pads and arms can be used. For example, one embodiment comprises as few as 2 or 3 sets of pads and arms, and other embodiments can include more than four sets of pads and arms, such as 5, 6, 7, or even 8 or more sets of pads and arms. The sets of pads and arms can be arranged symmetrically or asymmetrically around a central axis of the device.

The pads 202 couple to arms 204, which can be coupled to a central rod (not shown) that extends along a longitudinal axis of the device, through the handle 206, and is coupled to the expansion mechanism 208. The tissue contacting pads 202 can be designed to maintain stability through all dilation diameters. In some embodiments, the pads can have a saddle shape that maintains a constant waist size and pitch when expanded. In the embodiment shown in FIGS. 2a-2c, arms 204 have a scissor-like assembly, and include shafts 210 and 212 which rotate around pivot 214. Shafts 210 and 212 can be coupled to the pads and to the central rod. In FIGS. 2a-2c, expansion mechanism 208 is a mechanical knob configured to expand the arms of the vaginal dilation device. Manual rotation of the knob can cause the knob to move away from the handle and the central rod to move proximally into the handle, allowing shafts 210 and 212 to rotate around pivot 214 and push pads 202 radially outward from the central rod of the device. Since the arms 204 comprise a scissor-like assembly, the pads 202 can remain parallel to the device and to one another during expansion, which maintains the orientation of the pads to the tissue stable during dilation. Additionally, maintaining the pads in a parallel orientation can help keep the device in place in the tissue while maximizing force distribution along the pads. Rotation of the knob in the opposite direction can cause the central rod to move distally, causing the scissor-like assembly to collapse and moving the pads inwards towards the central rod.

FIGS. 2a and 2c depict the dilation device 200 as fitted within an adapter 230. FIG. 2a shows the dilation device 200 in a contracted configuration and FIG. 2c shows the dilation device in an expanded configuration. FIG. 2b also shows the dilation device in a substantially contracted configuration, and with a distal release 220 extending outwardly. FIG. 2b, for purposes of illustration, shows the dilation device 200 without the adapter 230 to present more clearly the proximal end of the dilation device 200. The adapter 230 includes a main body 238 and two arms extending from the main body 238 and forming recesses 234 that are sized to receive portions of a retention harness (not shown).

Although FIG. 1 and FIGS. 2a-2c show dilation devices with mechanically expanding arms to cause dilation, one being motorized and one being manually driven, it will be readily understood that other apparatus for dilating the vaginal canal of the patient may be employed with the retention devices described herein, including expanding balloons, expanding foam bodies, and any other mechanism suitable for delivering an outwardly directed and controlled expansion force. Such devices may be employed with the retention devices described herein for holding such alternate dilation devices in place within the vaginal canal for a period of time and at a location that is selected by the clinician to achieve a therapeutic effect.

For the procedures described herein, the dilation device 200 can be sized, shaped, and configured to penetrate approximately the first third, or 3-4 cm, of the vagina, and to gradually expand the vaginal introitus from a resting diameter of approximately 2 cm to a fully dilated diameter of approximately 10 cm. The dilation device 200 can be configured to expand from a compact configuration, as shown in FIG. 2a, to an expanded configuration, as shown in FIG. 2c. As the dilation device 200 dilates to the expanded configuration, as shown in FIGS. 2b-2c, pads 202 and arms 204 move radially outwards from the dilation device, causing the pads to separate from one another. In some embodiments, the maximum diameter of the pads in the expanded configuration can be approximately 10 cm. In one embodiment, the maximum outer diameter of the pads in the expanded configuration is approximately 8-12 cm.

Optionally, the vaginal dilation device 200 can comprise indicator(s) or gauge(s) 218. In the embodiment of FIGS. 2a-2c, the gauge 218 is disposed on handle 206, however the gauge can be disposed in any location on the dilation device, or even remotely from the dilation device such as on a display monitor. In some embodiments, the gauge gives the clinician an indication of the diameter of the dilation device across its pads 202. A user, such as a physician, can then use the gauge to know the amount of tissue dilation. Gauge 218 can comprise a simple binary readout (showing the diameter is above or below some threshold to continue dilating) or can have a scale showing the actual diameter of the dilation device, for example. Further optionally, the dilation device 200 may include a distal release mechanism configured to reduce the dilation device 200 from the expanded configuration to the closed configuration. The distal release mechanism may include a fetal body detection switch 220 that detects contact of the fetus against the distal release. The distal release can release the dilation device 200 from engagement with the vaginal wall and collapse the dilation device 200 into the closed configuration, and in any case into a reduce size configuration that prevents the dilation device 200 from obstructing passage of the baby and allows a clinician to remove the dilation device from the patient.

FIGS. 2d-2f depict an alternate embodiment of a dilation device 250 that is suitable for use with the systems and methods described herein. In particular, FIG. 2d depicts the dilation device 250 as having an adapter comprising integrated arms 252 that attach directly to and are integral with the distal end 254 of the dilation device 250. As depicted in FIG. 2d, the distal end 254 of the dilation device 250 includes the curved arms 252 that extend outwardly from the distal end 254 of the dilation device. As further depicted by FIG. 2d the distal end of the dilation device includes a user control 264 that may be operated by the patient, the clinician, or other party to operate the dilation device to transform between an expanded configuration and a collapsed configuration. To that end, FIG. 2e depicts the dilation device 250 as partially expanded, and shows that the pads 258 are partially extended from the dilation device 250. FIG. 2f depicts the arms 258 more fully extended and positioned outward from the dilation device 250 by the scissor arms 260 which in FIG. 2f are scissor mechanisms that drive the arms 258 outward from the dilation device 250.

Returning to FIG. 1, one can see that the vaginal dilation device 102 is held by the retention device 100 so that the dilation device 102 is fitted into and engaged with the adapter 110. In the embodiment depicted in FIG. 1 the distal end of the dilation device, which is the handle 206 of the device 200 in FIGS. 2a-2c, is fitted into the main body 116 of the adapter 110. In this depicted embodiment, the adapter 110 is a plastic housing sized to receive the handle of the dilation device 102. In one example, the adapter 110 is made of a plastic material suitable for use in medical applications and in one particular example the adapter 110 is made from Acrylonitrile Butadiene Styrene, more commonly known as ABS, or some other suitable thermoplastic polymer. In some embodiments the material is selected to allow for sterilization and reuse.

The adapter 110 can be sized and shaped so that the handle of the dilation device 102 “snap fits” into the adapter 110 and is held within the adapter by a frictional engagement. The frictional engagement allows the dilation device 102 to be removeable from the adapter 110 upon the application of a mechanical force, typically the manual hand force of the clinician, to overcome the frictional engagement and free the dilation device 102 from the adapter 110. Thus, the clinician can readily remove the dilation device from the adapter. This may be done once the therapeutic treatment is complete and the patient is ready for the final stages of labor and delivery. Additionally, this may be done during labor to allow the clinician to attach a fetal scalp monitor or deliver and place a catheter or take some other action that requires the clinician to have unobstructed access to the vaginal canal or cervix. In such cases where the access is needed for a temporary period, and before completion of the therapeutic procedure, the dilation device 102 may be removed from the adapter 110 temporarily and then replaced into the adapter 110 to allow the therapeutic procedure to continue. Optionally, this removal of the dilation device 102 can be done without removing the retention strap 118 from the patient. Further optionally, the removal of the dilation device 102 can be done after or as part of removing the retention strap from the patient.

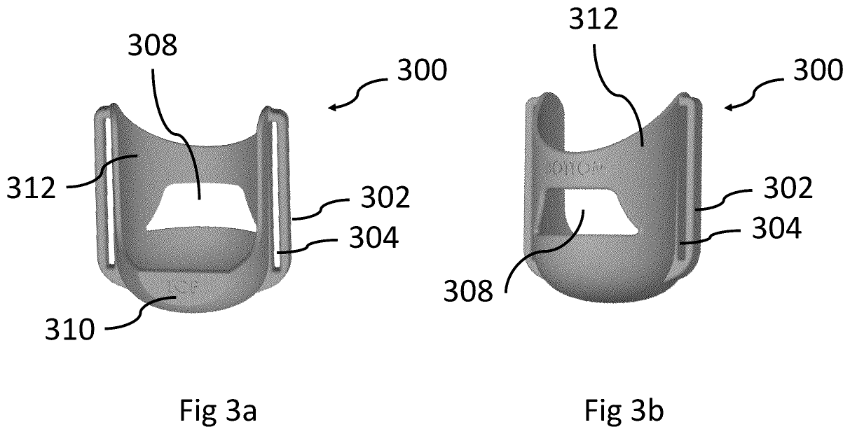

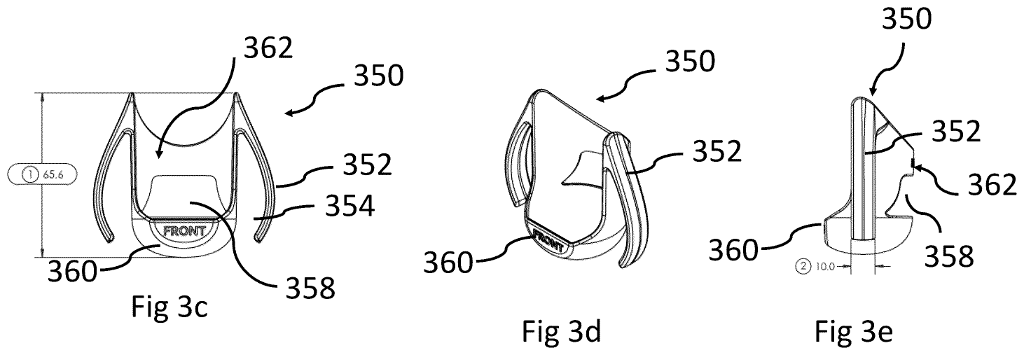

FIGS. 3a and 3b depict an alternate embodiment of an adapter 300 suitable for use with the retention device described herein. FIG. 3a depicts the adapter 300, the arms 302, the recess 304, the main body 310, the curved back wall 312, and a back wall opening 308. FIG. 3b depicts the adapter 300 from an alternate perspective showing in more detail the curved back wall 312 and back wall opening 308. In the embodiment depicted in FIGS. 3a and 3b the arms 302 extend outward from the main body 310 and extend along the longitudinal axis (not marked) of the adapter 300 to extend from the distal end of the adapter 300 to the proximal end of the adapter 300. Each arm 302 connects to the main body at a distal end and a proximal end of the arm 302. The offset between the arm 302 and the main body 310 forms the recess 304 which is sized and shaped to be suitable for receiving a portion of the retention harness (not shown). The adapter 300 itself is sized and shaped to receive the proximal end, typically the handle, of the dilation device such as the dilation device 102 depicted in FIG. 1. The proximal end of the dilation device can snap fit into the adapter 300 so that the handle of the dilation device 102 snaps into the adapter 300 and fits within the recess of the main body 310 formed by the curved back wall 312, with the curved back wall 312 following the curve of the handle of the dilation device 102. The face opposite the curved back wall 312 is open to allow access to user interface controls placed on the handle 108 of the dilation device 102. The back wall opening 308 allows for the main body 310 of the adapter 300 to flex and thereby more easily receive and snap onto the handle 208 of the dilation device 200. Additionally, the back wall opening 308 allows the clinician to access the handle 208 while in situ in the adapter 300 and thereby more easily press the handle 208 to push the handle out of and release the dilation device 200 from the adapter 300. In certain embodiments the dilation device may have grips, such as grips formed of a silicon material and placed on one side of the distal end of the dilation device. The silicon grips allow for a clinician to form a more reliable manual grasp of the dilation device. The opening 308 in the backwall of the adapter 300 allows the clinician to grasp the silicon grips placed on the rear side of the distal end of the dilation device to create a better grip in the dilation device even when the dilation device is fitted within the adapter. Optionally, the adapter 300 may include labels, such as the depicted Front and Back to allow the suer to more readily know which side of the adapter 300 is the front and thereby more easily insert the dilation device into the adapter correctly.

FIGS. 3c-3e depict a further alternate embodiment of an adapter 350 suitable for use with the retention device described herein. FIGS. 3c-3e depict the adapter 350, the arms 352, the recess 354, the main body 360, the curved back wall 362, and a back wall opening 358. FIGS. 3c-3e depict the adapter 350 from alternate perspectives to show in more detail certain features, such as the curved back wall 362 and back wall opening 358. In the embodiment depicted in FIGS. 3c-3d the arms 352 are shown as curved to have an arc that gives the outline of the adapter a convex appearance. The arms 352 attach to the main body 360 at the proximal end of the adapter 350 and are open at the distal end. The recess 354 is the space between the arms 352 and the main body 360. The arms 352 extend outwardly from the main body 360 and extend along the longitudinal axis (not marked) of the adapter 350 so as to extend from the distal end of the adapter 350 to the proximal end of the adapter 350.

The adapters 300 and 350 may be made of plastic, such as ABS, and typically will be a plastic body with enough elasticity to snap fit around the handle of the dilation device. However, other materials may be employed including fiberglass, aluminum or any other material suitable for the application of securing to the dilation device and connecting to a retention harness. In the embodiment of FIGS. 3a-3e the adapter 350 is about 65.6 mm in height and the width of the adapter from arm 352 to arm 352 is about 72 mm. Each arm 352 is about 10.00 mm in width.

The adapters 300 and 350 snap fit onto the handle of the dilation device. However, in other embodiments, the adapter may have a clasp, screw or other mechanical connector to join the adapters 300 and 350 to the dilation device and the method for securing the adapter to the dilation device may vary with the application including whether the adapter is to be easily and quickly released from engagement with the dilation device or whether the adapter is to have a more secure connection to the dilation device. The adapter 300 depicted in FIGS. 3a-d are two examples of adapters suitable for use with the retention devices and methods described herein. For example, in alternative embodiments, the adapter may include buttons for forming a mechanical connection to the retention harness. The buttons may connect to the main body of the adapter and may be snap fastener buttons of the type used to attach to fabric material and often called fabric snaps. In such cases, the adapter can have one side of the snap fastener secured to the main body of the adapter and the other side of the snap fastener may be attached to the fabric of the retention harness. Snapping the adapter to the retention harness can releasably secure the adapter to the retention harness. In other alternate embodiments, the adapter may include arms that are permanently secured to the retention harness, such as by sewing the arms of the adapter into fabric pockets formed into the retention harness.

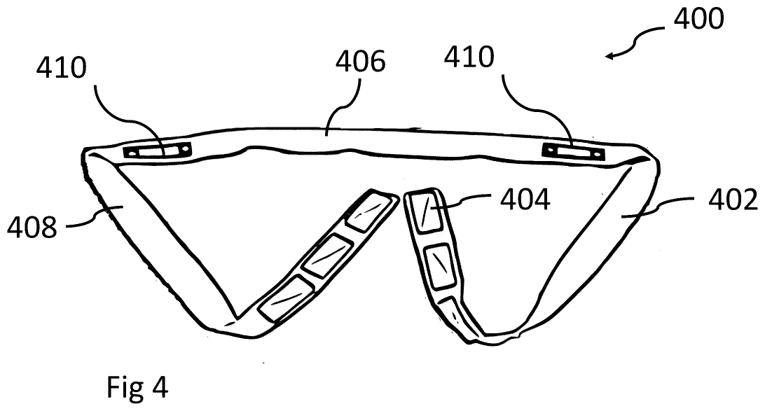

FIG. 4 depicts one embodiment of a retention harness 400 for use with an adapter, such as the adapters 300 and 350 of FIGS. 3a-c. The retention harness 400 in this embodiment is a strip of flexible material, often fabric and often elastic. The retention harness 400 is typically sized to fit around the patient either directly to the body of the patient or about the clothing and gown worn by the patient. The depicted retention harness 400 secures to the patient at a location that facilitates connecting the retention harness 400 to an adapter so that the adapter is positioned to maintain the dilation device in a therapeutically effective location.

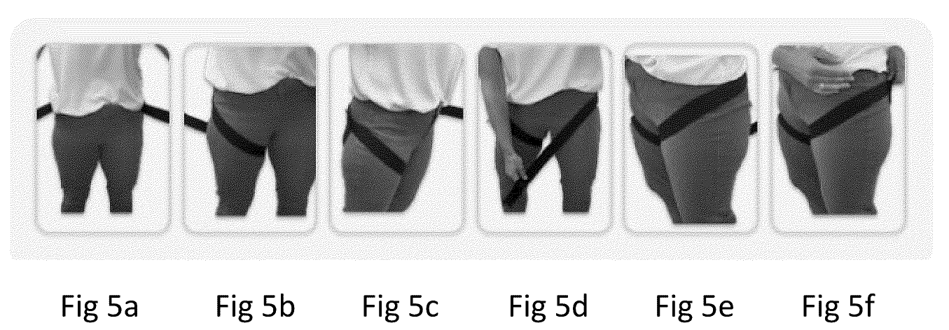

The depicted retention harness 400 is made of flexible and elastic material, such as an clastic and stretchable polyurethane fabric, some of which is sold under the brand name Spandex®. The retention harness 400 of FIG. 4 has a length, from end to end, that is sufficient to wrap around both thighs of the patient being treated. The retention harness 400 may have a section that traverses the thigh of the patient at a location superior the pubic arch of the patient and have securing features 402 that are shaped and sized to engage with the attachment features of the adapter. The retention harness 400 has a waist strap 406 that is sized to wrap around the back of the patient at their waist. This can be seen in FIG. 5a where the retention harness is shown wrapped around the waist of a patient model, such that the waist strap 406 extends across the back waist of the model. The retention harness 400 has two thigh wrap sections 402 and 408, each of which can be wrapped around a thigh of the patient. Releasable attachment mechanisms are provided by elements 404 and 410, which in this case are the respective parts of a hook and loop fastener.

As depicted in FIG. 4, the thigh strap sections 402 and 408 and waist strap 406 include hook and loop fastener elements 404 and 410 for allowing the one or more thigh straps 402 and 408 to form a closed loop around one or more thighs of the patient. This can be seen in FIGS. 5b-5f that show the thigh straps of the retention harness being secured around the thighs of the model and then secured in place to the waist strap 406 which is at the back of the model patients waist. In this depicted example the thigh straps 402 and 408 are secured to the waist strap 406 by a hook and loop fastener. In particular, FIG. 5a shows a model arranging a retention strap, such as the retention strap 400, at the back of their waist at or superior to their pelvis and superior to their pubic arch. In FIG. 5b, the model has wrapped the right portion of the retention strap, the thigh strap 408, about their right thigh passing the end of the thigh strap 408 through their crotch and back over and against their gluteus maximus. FIG. 5c shows, from a side perspective, the attachment of the end of the thigh strap 408 that has the hook element 404, to a corresponding loop fastener, in this case 410, which is not shown but is positioned at the upper waist of the model and arranged for engaging with the hook element 404. FIG. 5d depicts the model similarly securing the retention strap 400 about the models left thigh and FIGS. 5e and 5f show, from a side view perspective, attachment of the end of the thigh strap 402 with the hook fastener 404 to the loop fastener 410 on the waist strap 406 at the rear section of the retention harness 400 located at the back waist of the model.

The hook and loop fastener elements 404 and 410 can be engaged by being pressed together such that the hook element 404 is pressed firmly against the loop element 410 until hook and loop engagement takes place. The size of the hook and loop fastener 404 and 410 may be selected so that the engagement of the hook element with the loop element is sufficiently robust to resist separation when a force is applied to the retention harness pulling the loop element 404 away from the hook element 410 and that applied force is the type of force that typically is produced by the patient moving about during the labor process. In this way the retention harness 400 can stay fixed about the waist and thighs of the patient even as the patient moves about. However, the binding force of the hook and loop fastener 404 and 410 may be selected so that it will release upon a force of the type that can be applied by a clinician, seeking to remove intentionally the retention harness 400 from the patient. Such force can be the type of force an obstetrician typically applies to remove a well secured strap, held by hook and loop fastener, from a patient. Typically, it is the force generated by strong tearing pull of the type that generates a shearing force that can rip the hooks from the loop.

The retention harness may be made from an elastic material to facilitate stretching. The amount of elasticity is selected to provide sufficient elasticity to allow for good attachment to the patient, so that the elastic thigh straps 406 and 408 can elastically wrap around the thighs of the patient and optionally provide an elastically contracting force that pulls the thigh straps in a superior direction and toward the vaginal canal of the patient. As contractions during labor may apply an outwardly directed force to the dilation device, the elasticity of the retention strap should be selected to resist this force sufficiently to prevent dislodging of the dilation device from the patient. In certain embodiments the retention harness may be made of a medical grade fabric. The width of the harness may be selected to balance patient comfort and strength. In one embodiment, the width of the harness is approximately 1.5 inches wide, but other widths may be selected and the width selected will depend upon the application at hand, including the expected body type of the patient. In certain embodiments, the hook and loop fastener attachment is disposed at select locations on the harness and at those select locations the retention harness can be secured by securing the hooks into the loops of the fastener. However, in certain alternate embodiments, one side of the retention harness is fully, or essentially fully, covered in either the loop or hook of the hook and loop fastener to allow for attachment at any location along the retention harness. Further, in other embodiments other fasteners may be employed including buttons, zippers, snaps, clips, buckles or any fastener suitable for attaching the material of the retention harness in such a way as to allow the retention harness to secure to the body of the patient. If necessary, the clinician can even form knots with the straps in order to hold the retention harness in place and the choice of fastener employed will depend in part on the preference of the clinician who may choose one retention harness over another based on the fastening system employed by the harness.

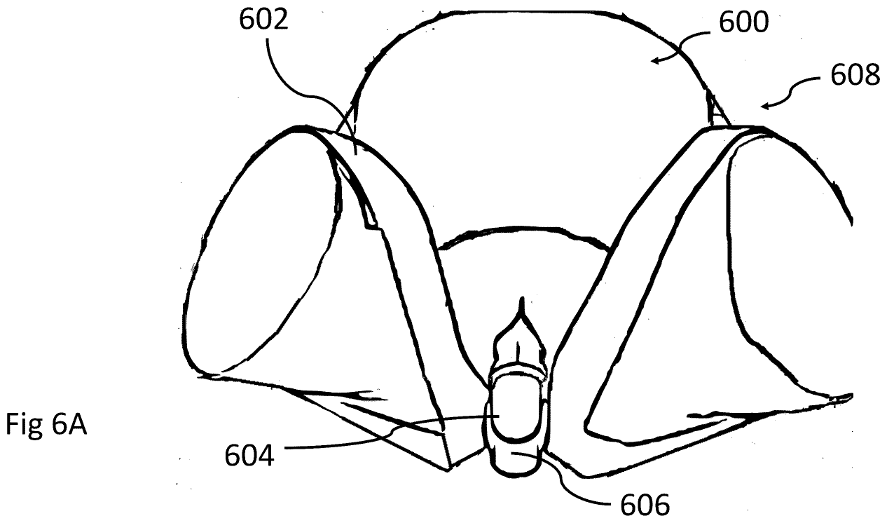

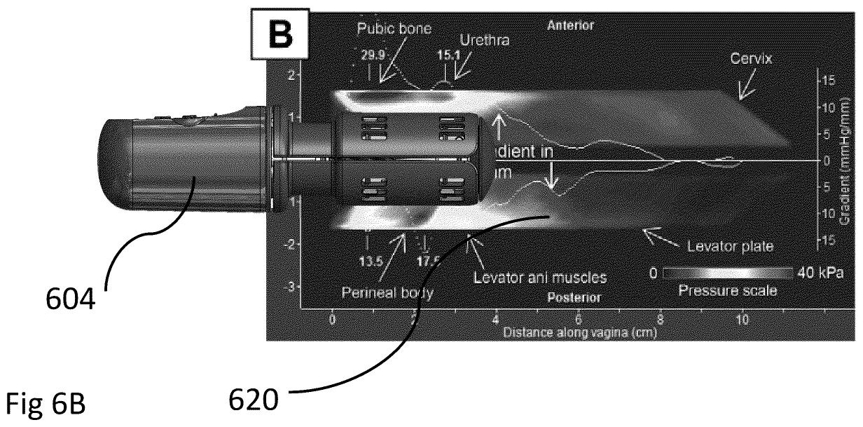

FIG. 6a depicts a partial model of a patient having a conventional habitus and being therapeutically treated with the retention device described herein. In particular, FIG. 6a depicts a partial model 600 of a patient showing the retention device 608 including the retention harness 602, the adapter 606 and the dilation device 604. In the image of FIG. 6a the retention device adapter 606 is attached to the proximal end of the dilation device 604 and the retention harness 602 is attached to the two arms (not shown as they are covered by the retention harness) of the adapter 606. The retention device 608 retains the dilation device within the vaginal canal of the patient at a location useful for having a therapeutic effect on the patient. As positioned, the retention harness 602 restricts the adapter 606 from moving proximally away from the birth canal of the patient, even as contractions and the motions of labor tend to push objects from the birth canal and even as the patient moves about as is typical for a patient in the labor process. In FIG. 6a, the adapter 606 and dilation device 640 may be similar to the adapter and dilation device depicted in FIG. 1 which includes a motorized dilation device. The adapter 606 is shown as engaged with a proximal end of the dilation device 640 and the distal end of the dilation device 604 extends outwardly from the adapter 606 and into the vaginal canal of the patient. The retention harness 602 may be similar to the retention harness 400 depicted in FIG. 4 and may be made of a flexible and elastic material. The retention strap 602 has a length sufficient to wrap around the upper waist and both thighs of the patient and has a section that traverses the thigh of the patient at a location superior the pubic arch of the patient. This is understood to provide mechanical support for the retention strap 602 to allow the retention strap 602 to be able to apply a superior directed force in opposition to the contractive forces arising during labor and tending to push the dilation device 604 proximally and from the patient. The thigh straps of the retention harness 602 secure into the recesses (not shown) between the arms of the adapter and the main body of the adapter such that the retention harness 602 restricts the adapter 606 from moving proximally away from the birth canal of the patient during treatment. FIG. 6a illustrates that the retention device 600 can hold the dilation device 604 in position to have a therapeutic effect for the patient. The clinician can use the retention device 608 to maintain the dilation device 604 in that therapeutically effective location for a time period sufficient to have a therapeutic effect.

FIG. 6b depicts an MRI model of the vaginal canal of the patient 600 and the location at which the dilation device 604 may be maintained by the retention device 608. As depicted in FIG. 6b, the retention device 608 holds the dilation device 604 so that the proximal end of the dilation device 604 is positioned within the vaginal canal of the patient 600. In FIG. 6b the dilation device 604 is positioned within the vaginal canal so as to be proximate the pubic bone and applying an expansive force proximate the pubic bone. As shown in FIG. 6b, the dilation device is held in this example proximate the patient's urethra, across the perineal body and levator ani muscles and maintained distal from the cervix. In one embodiment, the methods described herein employ the retention device 608 to maintain a dilation device in the vaginal canal and proximate the perineal body and across levator ani muscles to provide a therapeutic expansive therapy force to this complex of muscles and to this body during the labor process.

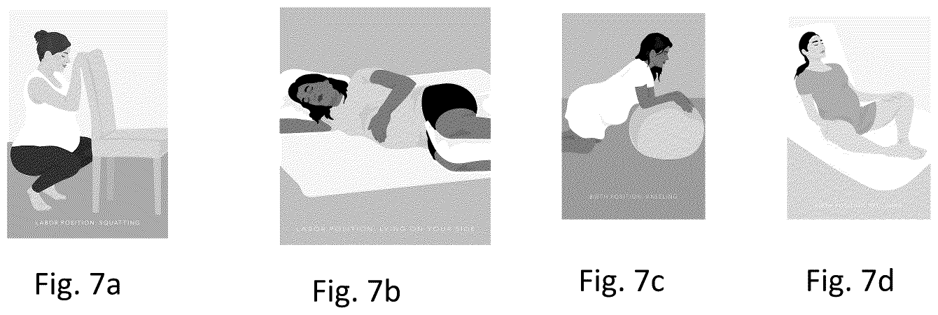

The retention devices described herein may be wearable by the patient to maintain the dilation device in place, even as the patient moves through some common labor positions. FIGS. 7a-7d depict several examples of labor positions that the patient may take during a labor and delivery process. As can be seen from FIGS. 7a through 7d, the patient may rise from the hospital bed during the process of labor and orient themselves in different positions for the purposes of improving comfort, encouraging the labor process, or for other reasons. FIG. 7A shows the patient in a squatting position. FIG. 7B shows the patient lying on their side with a pillow between their legs and under their head. FIG. 7C depicts the patient on their knees with their upper torso supported by a support ball and FIG. 7D shows the patient reclined in bed, with the bed raised partially to support the patient's back. In all four illustrations, it can be seen that the labor process is one that is quite physical and active and benefits from a retention device that can maintain a dilation device in a therapeutic location and for a period of time that is therapeutically useful even while the patient moves about the room and changes their position in ways relevant to the birthing procedure. To this end, the retention harness, such as the retention harness 400 depicted in FIG. 4 is typically soft and flexible and comfortable so that the patient can wear it with comfort during the labor process even as the patient moves about the room and positions themselves in different orientations. Additionally, the retention harness, such as the retention harness 400 depicted in FIG. 4, can be secured to the body of the patient with sufficient strength so that the retention harness does not easily release from the patient even as the patient moves into a squatting position or lying position. In the embodiment depicted in FIG. 4 the retention harness 400 includes hook and loop fasteners that can be used to secure the retention harness to the patient with sufficient strength to resist separating from the patient even as the patient moves about the room and changes their position.

The retention devices described herein may hold the dilation device in place for the duration of the therapeutic procedure. For example, the retention device may hold the dilation device in the vaginal canal for a period of time sufficient to achieve a therapeutic effect. Typically, that therapeutic effect is the controlled and gradual stretching of the vaginal canal an amount sufficient to facilitate subsequent delivery of the baby with reduced injury to the patient. To this end, the dilation device can incrementally increase in diameter, consequently increasing the size of the vaginal canal. The incremental increase may be gradual enough to allow for tissue to expand without tearing or otherwise being damaged. Tissue can be expanded gradually such that the tissue material retains sufficient elasticity during the expansion to avoid tearing and damage. Though not to be limited or restrained by theory, the tissue wall of the vaginal canal is part of the birth canal and anatomically generally capable of passing the baby. However, as tissue of the pelvic floor and the vaginal canal can, during many birthing procedures, be subjected to sudden outward force as contractions push the head and shoulders of the baby rapidly through the birth canal and across the pelvic floor. As the baby descends through the birth canal, the fetal head is pushed by contractions passed the cervix and through the vaginal canal, which forms a passage through the pelvic floor and the complex of muscles and ligaments that make up the pelvic floor. The fetal head may be large and the area of the average fetal head, in the plane of minimal diameters, measures 70-100 cm² and generally has a head circumference of 300-350 mm, requiring marked distension and deformation of the levator complex, the complex of muscles around the ani levator. MRI-based computer modeling has shown that the most inferior and medial parts of the levator complex must increase in length by more than three times during crowning of the fetal head. Given this degree of acute distension, it is remarkable that severe muscular trauma is not more prevalent, as such stretch is commonly thought to be well beyond the approximately 150% elastic limits of tissue strain. In any case, this rapid outward expansion that occurs during delivery may occur too quickly for the untreated tissue. The therapeutic devices described herein may give the tissue of the birth canal and the pelvic floor, including the levator complex, sufficient time to adjust by allowing the tissue to recruit sufficient fluid and blood flow to allow the tissue to expand the needed amount, such as for example having the vaginal canal expand to 10 cm (or 100 mm) in diameter. By allowing the vaginal wall and pelvic floor to expand a priori the passing of the baby, the tissue wall of the vaginal canal and the tissue of the pelvic floor are prepared to expand to allow the passage of the baby without tearing and damage or with reduced tearing and damage.

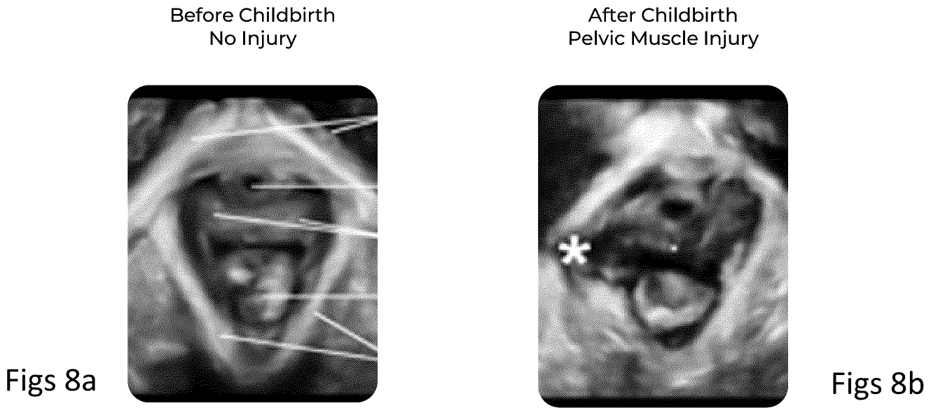

FIGS. 8a and 8a are magnetic image resonance (MRI) images of actual patients not treated with the systems and methods described herein. FIG. 8A illustrates the healthy and uninjured levator complex of a patient, while FIG. 8b illustrates an injured patient. Comparison of the images in FIGS. 8a and 8b illustrate that substantial injury of the levator complex can arise during labor. In FIG. 8b it is shown that tissue is torn from the bone structure of the pelvis and the patient has suffered a pelvic muscle injury during labor and passage of the fetus through the vaginal canal. The illustrated example injury is substantial and may resist complete healing. Such injuries to the complex that provide the pelvic floor may arise during labor and delivery and can lead to additional conditions, including prolapse of the bladder and lack of support for the sphincters of the patient, causing incontinence, either permanent or intermittent. The systems and methods described herein are designed to reduce or eliminate such injury to the pelvic floor during labor.

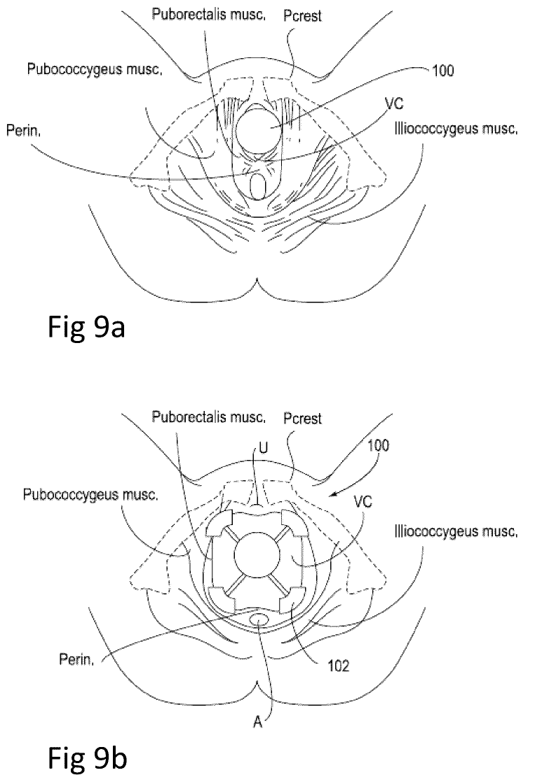

FIGS. 9a and 9b illustrate the therapeutic effect of a vaginal dilation device employed to therapeutically expand the tissue of the birth canal. In FIGS. 9a and 9b there is depicted one example of a vaginal dilation device 900 of the type used with the retention devices described herein. In FIG. 9a, the dilation device 900 is shown inserted into the vagina in a closed configuration. The outer diameter of the dilation device 900 in the closed configuration can be less than 4 cm, and this may be used during initial insertion to provide placement with sufficient patient comfort. FIG. 9b illustrates dilation device 900 in an expanded configuration. The outer diameter of the dilation device 900 in the expanded configuration can be up to approximately 10 cm. The device can be inserted into the patient any time during the first phase of labor, and the treating clinician can select the time to being treatment. On average, a woman spends 14 hours in a hospital until the second phase of labor begins. After insertion the dilation device 900 is incrementally expanded, such as over one to three hours, and the dilation device 900 is expanded as thought best by the clinician and in some therapies, the device is expanded to about 10 cm in diameter. To provide a more effective treatment with the dilation device 900, the clinician may employ a retention device to maintain the dilation device 900 within the vaginal canal during the labor process. The retention device resists contractual forces, and forces are rising during repositioning of the patient during labor, applied to the dilation device 900 and operates to maintain the dilation device 900 at a therapeutically effective location for therapeutically effective period of time.

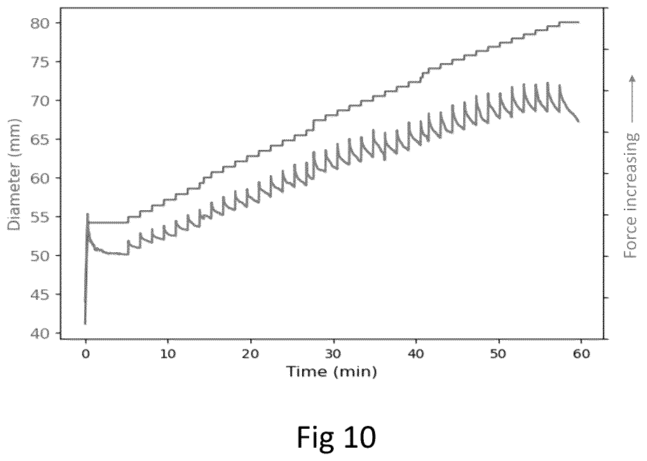

FIG. 10 is a chart illustrating a method of dilating vaginal tissue that may be used, at least during parts, with the retention devices described herein. The right y-axis indicates force applied by the dilation device to the vaginal canal of the patient, the left y-axis indicates the expanding diameter of the dilation device and the x-axis indicates time in minutes.

Referring to the chart, the diameter of vaginal tissue was, for this treatment, about 5.5 cm and was increased to about 8 cm over a period of about 1 hour (60 minutes). The amount of force applied to tissue was increased gradually to provide for increased diameter while avoiding tissue damage. For example, initially a force was applied by the dilation device to dilate the tissue to about 5.5 cm (or 55 mm). After the initial dilation, the tissue was relaxed, and it can be seen that the measured force applied by the device to the tissue decreased for approximately 4 minutes. This suggests the tissue elastically adjusted to the initially applied force as one can see that the increased diameter of 5.5 cm remained, even as applied force dropped. Next, a gradually increasing and incremental force (incrementing over about 1-minute periods) was applied by the device to dilate the tissue to 5.6 cm, followed by a time period of no additional dilation which allowed the tissue to relax and the measured applied force to decrease. This incremental process was repeated until the maximum dilation of about 8.0 cm (80 mm) was achieved.

It should be understood that these values are merely explanatory, as every woman's body and tissue response to treatment may be different. In general, however, a method of dilating vaginal tissue can comprise inserting a vaginal dilation device into the vagina, measuring a force applied by the vaginal dilation device to the vagina, dilating the vagina with the vaginal dilation device, and pausing or stopping dilation of the vagina with the vaginal dilation device when the force applied by the vaginal dilation device to the vagina increases to a first force threshold. In some embodiments, the method further increases measuring a diameter of the vagina with the vaginal dilation device. Next, the method can include resuming dilation of the vagina when the force applied by the vaginal dilation device to the vagina decreases to a second force threshold. For example, a user or controller may determine that the tissue has relaxed enough that it is time to begin dilating the vaginal tissue to a larger diameter. In some embodiments, dilation is resumed until the force applied by the vaginal dilation device increases to the first force threshold, or alternatively, until the force applied increases to a third force threshold larger than the first force threshold. In some embodiments, the first, second, and third force thresholds range from approximately less than 8 lbs. of force.

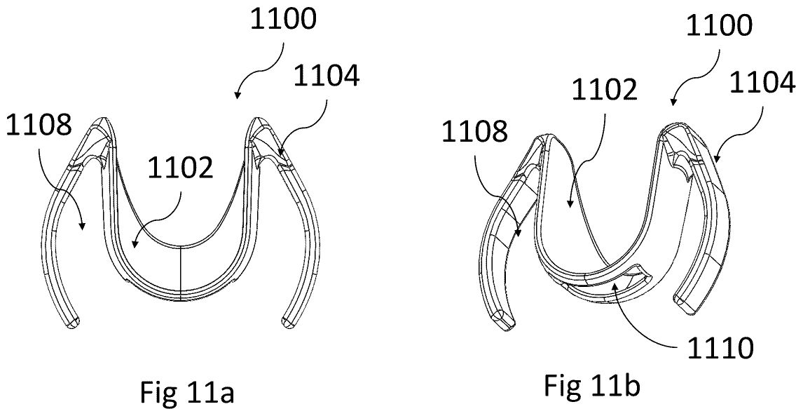

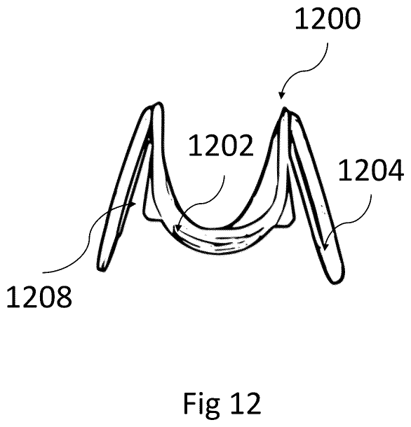

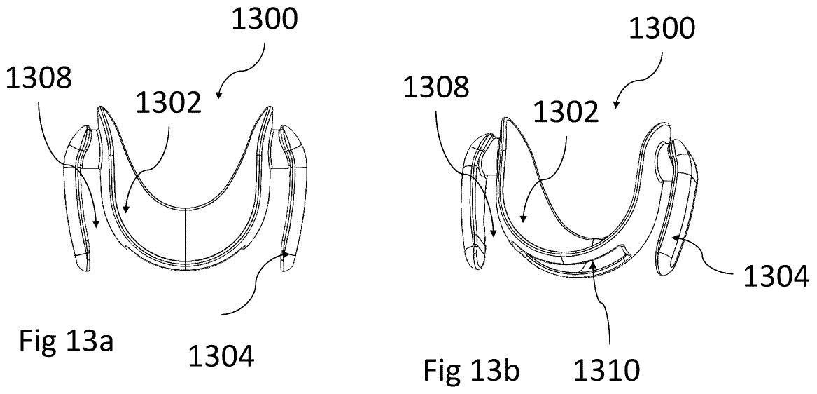

FIGS. 11a and 11b depict from different perspectives a further alternate embodiment of an adapter 1100 that includes a main body 1102, and a pair of curved arms 1104. The curved arms 1104 form a curved recess 1108 between the arms 1104 and the main body 1102. The curved recess 1108 is sized to receive portions of the retention harness for securing the adapter 1100 to the retention harness. An opening 1110 allows for access to the dilation device and can facilitate removal of the dilation device from the adapter 1100. FIG. 12 depicts a further alternative embodiment of an adapter 1200. The depicted adapter 1200 includes a main body 1202 and arms 1204. The arms extend at an acute angle from the main body 1204 and provide a recess 1208 between the arms 1202 and the main body 1204. FIGS. 13a and 13b depict from different perspectives yet another embodiment of an adapter 1300 that includes to arms 1304 extending outwardly from the main body 1302 and providing a recess 1308 into which the retention harness can fit for securing the adapter to the retention harness. An opening 1310 allows for access to the dilation device and can facilitate removal of the dilation device from the adapter 1300. It will be understood by those of skill in the art that different configurations of an adapter may be employed by the retention devices and methods described herein without departing from the scope of the invention.

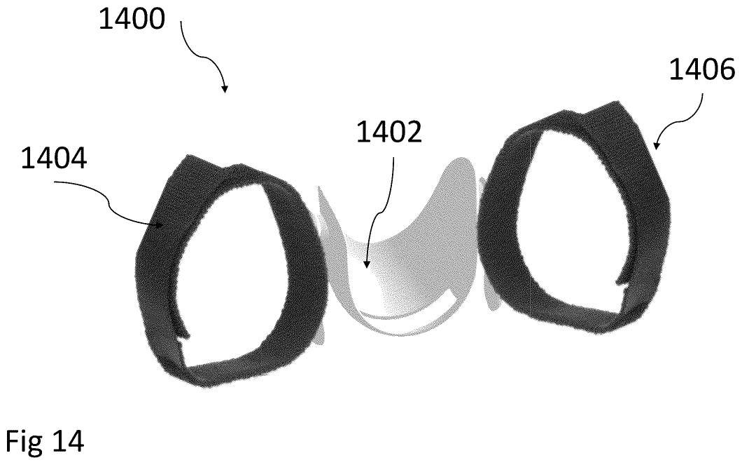

FIG. 14 depicts a further alternative embodiment of the retention systems described herein. In particular, FIG. 14 depicts a retention harness 1400 that includes an adapter 1402 and two fabric loops 1404 and 1406. The adapter 1402 fits between the fabric loops and, as illustrated in FIG. 14, the adapter 1402 may include two arms that extend outwardly from a main body. In this example of this alternative embodiment of a retention system, the adapter 1402 is comparable to the adapter depicted in FIG. 13. In any case, the retention harness 1400 in this embodiment includes two fabric loops. The fabric loops 1404 and 1406 may each be formed from a strip of an elastic flexible material that includes hook and loop fasteners. To this end, FIG. 14 illustrates each loop 1404 and 1406 as a strip of fabric that has been formed into a loop, with the ends of the fabric strip overlapping. The hook and loop fastener (not shown) is arranged on each fabric strip such that the two ends of the fabric strip may overlap as shown and join together to form the loops depicted in FIG. 14.

In use, each of the loops 1402 and 1404 may be wrapped around a respective thigh of the patient and secured sufficiently tightly to prevent the fabric loop from sliding along the length of the patient's thigh during labor. The fabric loops secured to the patient thigh join to the arms of the adapter 1402 by sliding into the recess formed between the adapter's main body and the adapter arms. Once in place, the two loops 1404 and 1406 act as a retention harness that maintains the adapter 1402 in place so that a dilation device (not shown) fitted within the adapter 1402 may be maintained at a therapeutically effective location for a therapeutically effective period of time. The loops around the thighs may be disposed high enough on the habitus of the patient to be superior to the pubic arch of the patient.

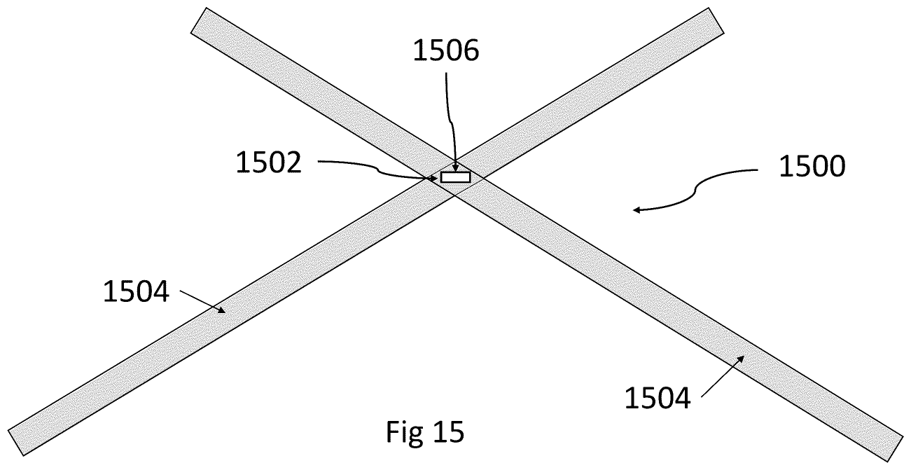

FIG. 15 depicts a further alternate embodiment of a retention harness 1500. The retention harness 1500 comprises two strips 1504 of a flexible material, in one case an elastic and flexible fabric such as for example a polyurethane material of the type sold under the brand name Spandex®. As depicted in FIG. 15 the two strips 1504 are arranged to form an overlap region 1502 and are connected at the overlap region 1502 such that the two strips 1504 are joined to form an X-shape. The connection at the overlap region 1502 in the depicted embodiment is formed by sewing together the two separate strips 1504 such that they are joined together at a location that divides each strip 1504 into a long portion and a short portion. In this embodiment, the, a receiving slot adapter 1506 is formed at the overlap portion 1502 and provides an opening through both straps 1504. The receiving slot adapter 1506 is sized to receive a portion of the proximal end of a dilation device, for releasably connecting a dilation device to the retention harness 1500.

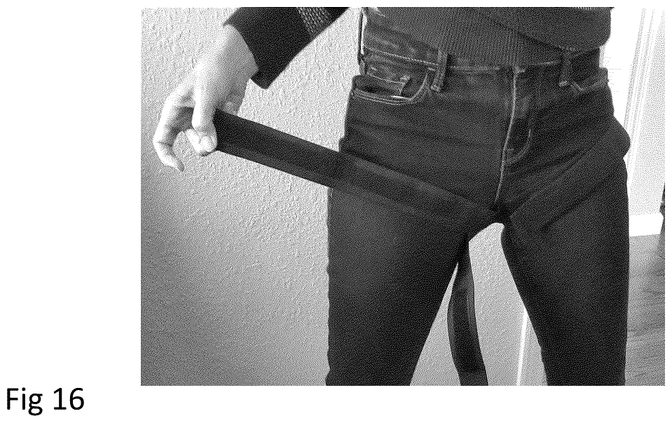

FIG. 16 depicts a model applying the retention harness 1500 to their body. As depicted in FIG. 16 the retention harness 1500 is positioned so that the connected overlap region 1502 fits proximate and proximal the pubic arch of the model patient and a long portion and the short portion of the strap are brought together and joined such that the long portion and the short portion form a loop that wraps around the thigh of the model patient. To join the long portion and the short portion of the strap, the retention harness 1500 may use a hook and loop fastener of the type commonly employed for joining fabric materials, or any suitable fastener.

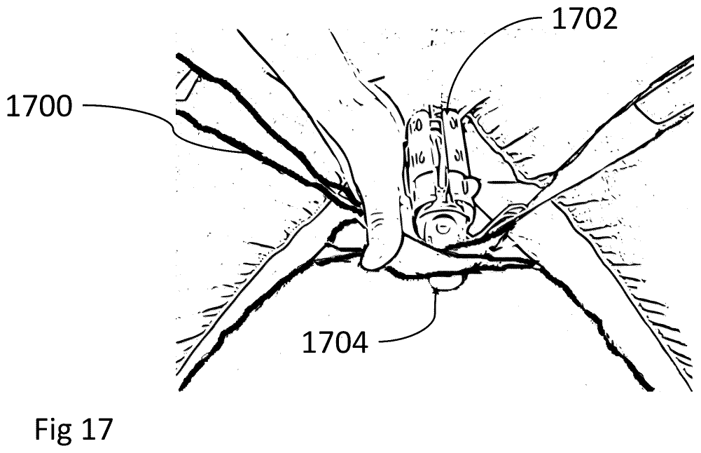

FIG. 17 depicts a retention harness 1700 of the type depicted in FIGS. 15 and 16, and that is arranged about the thighs of a model patient (wearing pants) and having a dilation device 1702, in a contracted form, fitted into a receiving slot adapter in the retention harness 1700, of type similar to the receiving slot adapter 1506 depicted in FIG. 15. As depicted in FIG. 17, the dilation device 1702 may have a portion of its proximal end fit against and slide into and through the receiving slot adapter to releasably join to the retention harness 1700. The receiving slot adapter provides engagement with the retention harness 1700 without the use of a separate plastic adapter. In this embodiment, the retention harness employs a sufficiently robust fabric material to provide an opening through the overlapped strips of fabric slot, where the opening is sufficiently strong to resist tearing forces generated in response to downward movement of the dilation device 1702.

The embodiments described herein are not to be understood as limiting and various modifications and additions may be applied to these embodiments without departing from the scope hereof, including retention systems that permanently connect an adapter to a retention harness, such as by sewing the adapter to the retention harness. Additionally, the retention harness may have several alternate shapes, and for example an alternative retention harnesses may be shaped as undergarments that may be worn by the patient during labor and which will retain a dilation device in a therapeutically effective location for a therapeutically effective time. In addition, retention harnesses may be employed that may attach to other locations on the body of the patient, such as fabric straps that wrap about upper portions of the patient's body, such as about the shoulders of the patient. Further additionally, although the retention harness has been described as fabric straps for surrounding and securing to portions of the patient's body, in alternate embodiments, adhesive may be employed to secure fabric straps directly to the patient's body. For example, an adhesive may be applied to a surface of a fabric strap. The adhesive bearing strap, much like an adhesive bandage, may attach to the patient's body upon the application of some force. This may cause the fabric strap to adhere to the patient. An adapter can then be joined to the adhered fabric strap.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A device for reducing injury to a patient during a labor and delivery procedure, comprising:
an adapter shaped to engage with a proximal end of a vaginal canal dilation device and to allow the distal end of the dilation device to extend distally from the adapter and having mounts for receiving a retention harness, and
a retention harness made of flexible material and having a length sufficient to wrap around a thigh of the patient and having a section that traverses the thigh of the patient at a location superior the pubic arch of the patient and having securing features that are shaped to engage with the attachment features of the adapter,
whereby the retention harness restricts the adapter from moving proximally away from the birth canal of the patient.

2. The device of claim 1, wherein the retention harness comprises one or more straps of elastic material.

3. The device of claim 2, wherein the one or more straps of elastic material include a fastener to allowing the one or more straps to form a closed loop around the body of a patient.

4. The device of claim 2, wherein the one or more straps include a hook and loop fastener for allowing the one or more straps to form a closed loop around one or more thighs of the patient.

5. The device of claim 2 wherein the retention harness comprises one strap of flexible material sized to wrap around the waist and around the thighs of the patient to form a harness having two sections of strap positioned between the thighs of the patient and superior to the pubic arch of the patient.

6. The device of claim 1, wherein the retention harness has a release that disconnects the harness from the patient or the adapter upon application of a proximally directed force that exceeds an expected force of a baby traveling proximally through the birth canal during delivery.

7. The device of claim 6 wherein the release comprises hook and loop fastener arranged on the strap to provide a frangible release for disconnecting the harness from the patient or the adapter.

8. The device of claim 1, wherein the mounts comprise arms extending radially and proximally from the adapter to form a bend that is sized to engage with the retention harness.

9. The device of claim 6 wherein the arms are formed as rails that attach to the sides of the adapter and provide an interior slot for receiving the retention harness.

10. The device of claim 1, wherein the adapter comprises a plastic body shaped to fit around a handle of the dilation device.

11. The device of claim 1, wherein the adapter is an integral part of the vaginal canal dilation device.

12. A method for reducing tissue injury during a labor and delivery procedure, comprising
providing a vaginal canal dilation device of the type used to provide a therapeutic expansive force during at least a portion of the labor procedure,
providing a wearable retention harness having a length sufficient to wrap about a waist and at least one thigh of the patient,
securing the retention harness to the patient by wrapping the harness around the waist and at least one thigh of the patient so that the harness secures to the patient at a first location proximate the pubic arch and at a second location superior the pubic arch, and
securing the dilation device into the retention harness at the second location and disposing at least a portion of the dilation device within the vaginal canal and securing a different portion of the dilation device to the retention harness such that the retention harness resists unwanted exit of the dilation device from the vaginal canal.

13. The method of claim 12 further comprising, providing
an adapter coupled to a proximal end of a vaginal canal dilation device to allow the distal end of the dilation device to extend distally from the adapter and having mounts for slidably receiving the retention harness.

14. The method of claim 13, wherein providing an adapter includes providing a removable adapter capable of releasably coupling to the proximal end of a vaginal canal dilation device.

15. The method of claim 13 wherein providing an adapter includes providing a vaginal canal dilation device having a proximal end with the mounts integrated therein.

16. The method of claim 12, wherein securing the retention harness to the patient includes employing a fastener to fasten a first section of the retention harness to a second section of the retention harness.

17. The method of claim 16, wherein employing a fastener includes employing at least one of a hook and loop fastener, a button, a zipper, a clips and a buckle.

18. The method of claim 12, wherein securing the vaginal canal dilation device into the retention harness at the second location and disposing at least a portion of the dilation device within the vaginal canal includes securing the retention harness to secure the dilation device at a location within the vaginal canal of the patient so that an expansion force may be applied to the levator ani muscles of the patient during the labor process.

19. The method of claim 16, further comprising maintaining the vaginal canal dilation device within the vaginal canal of the patient and applying an expansion force to a tissue wall of the vaginal canal for a period of time between 30 minutes and 120 minutes.

20. The method of claim 19, further comprising temporarily removing the vaginal canal dilation device from the vaginal canal during the period of time between 30 minutes and 120 minutes.
